(19) United States
(12) Patent Application Publication
(10) Pub. No.: US 2023/0045099 A1
(43) Pub. Date: Feb. 9, 2023

(54) MACHINE LEARNING TECHNIQUES FOR SIMULTANEOUS LIKELIHOOD PREDICTION AND CONDITIONAL CAUSE PREDICTION

(71) Applicant: Optum Technology, Inc., Eden Prairie, MN (US)

(72) Inventors: Sree Harsha ANKEM, Hyderabad (IN); Shyam Charan MALLENA, Hyderabad (IN); Ninad D. SATHAYE, Bangalore (IN); Gregory J. BOSS, Saginaw, MI (US); V Kishore AYYADEVARA, Hyderabad (IN); Aditya MADHURANTHAKAM, Hyderabad (IN)

(21) Appl. No.: 17/368,407

(22) Filed: Jul. 6, 2021

(51) Int. Cl. A61B 5/11; A61B 5/00; G06N 20/20; G06N 5/04; G06N 3/04

(52) U.S. Cl. CPC A61B 5/1117; A61B 5/7282; G06N 20/20; G06N 5/04; G06N 3/0454

(57) ABSTRACT

There is a need to accurately and dynamically predicting a probability for an event and a likely cause for the event prior to the event occurring using collected data from disparate data sources. This need can be addressed, for example, by generating an event prediction data object by utilizing an event prediction machine learning model, wherein the event prediction data object describes an event likelihood prediction and in an instance where the event likelihood prediction is an affirmative likelihood prediction, one or more fall cause predictions; and performing one or more prediction-based actions based at least in part on the event likelihood prediction.

### BACKGROUND

Various embodiments of the present invention address technical challenges related to accurately and dynamically predicting a probability for an event and a likely cause for the event prior to the event occurring using collected data from disparate data sources. In doing so, various embodiments of the present invention make important contributions to various existing predictive data analysis systems.

### BRIEF SUMMARY

In general, embodiments of the present invention provide methods, apparatuses, systems, computing devices, computing entities, and/or the like for dynamically generating a fall likelihood prediction for a user feature data object.

In accordance with one aspect, a method includes: generating, using the one or more processors and by utilizing a fall prediction machine learning model that is configured to process a user feature data object, a fall prediction data object, wherein: the fall prediction data object describes: (i) a fall likelihood prediction, and (ii) in an instance where the fall likelihood prediction is an affirmative true likelihood prediction, one or more fall cause predictions, the user feature data object comprises one or more numerical timeseries feature data fields, one or more categorical timeseries feature data fields, and one or more static feature data fields, and the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall prediction data object based at least in part at least in part on the numerical timeseries embedding, the categorical timeseries embedding, and the static embedding; and performing, using the one or more processors, one or more prediction-based actions based at least in part on the fall likelihood prediction.

In accordance with another aspect, an apparatus comprising at least one processor and at least one memory including program code, the at least one memory and the program code configured to, with the processor, cause the apparatus to at least: generate, using a fall prediction machine learning model that is configured to process a user feature data object, a fall prediction data object, wherein: the fall prediction data object describes: (i) a fall likelihood prediction, and (ii) in an instance where the fall likelihood prediction is an affirmative true likelihood prediction, one or more fall cause predictions, the user feature data object comprises one or more numerical timeseries feature data fields, one or more categorical timeseries feature data fields, and one or more static feature data fields, and the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall prediction data object based at least in part at least in part on the numerical timeseries embedding, the categorical timeseries embedding, and the static embedding; and perform, one or more prediction-based actions based at least in part on the fall likelihood prediction.

In accordance with yet another aspect, a computer program product computer program comprising at least one non-transitory computer-readable storage medium having computer-readable program code portions stored therein, the computer-readable program code portions configured to: generate, using a fall prediction machine learning model that is configured to process a user feature data object, a fall prediction data object, wherein: the fall prediction data object describes: (i) a fall likelihood prediction, and (ii) in an instance where the fall likelihood prediction is an affirmative true likelihood prediction, one or more fall cause predictions, the user feature data object comprises one or more numerical timeseries feature data fields, one or more categorical timeseries feature data fields, and one or more static feature data fields, and the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall prediction data object based at least in part at least in part on the numerical timeseries embedding, the categorical timeseries embedding, and the static embedding; and perform, one or more prediction-based actions based at least in part on the fall likelihood prediction.

In accordance with one aspect, a method includes: generating, using the one or more processors and by utilizing a fall prediction machine learning model that is configured to process a user feature data object, a fall prediction data object, wherein the fall prediction machine learning model is generated based at least in part on optimizing a custom loss model, the custom loss model comprises a fall likelihood component and a fall cause component, and the custom loss model is generated in accordance with a custom loss generation routine that comprises: identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications; generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications; for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object; generating the fall likelihood component based at least in part the fall likelihood loss values for the one or more training user feature data objects; and generating the fall cause component based at least in part on the fall cause loss values for the one or more training user feature data objects; and performing, using the one or more processors, one or more prediction-based actions based at least in part on the fall likelihood prediction.

In accordance with another aspect, an apparatus comprising at least one processor and at least one memory including program code, the at least one memory and the program code configured to, with the processor, cause the apparatus to at least generate, using a fall prediction machine learning model that is configured to process a user feature data object, a fall prediction data object, wherein: the fall prediction machine learning model is generated based at least in part on optimizing a custom loss model, the custom loss model comprises a fall likelihood component and a fall cause component, and the custom loss model is generated in accordance with a custom loss generation routine that comprises: identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications; generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications; for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object; generating the fall likelihood component based at least in part the fall likelihood loss values for the one or more training user feature data objects; and generating the fall cause component based at least in part on the fall cause loss values for the one or more training user feature data objects; and perform one or more prediction-based actions based at least in part on the fall likelihood prediction.

In accordance with yet another aspect, a computer program product computer program comprising at least one non-transitory computer-readable storage medium having computer-readable program code portions stored therein, the computer-readable program code portions configured to: generate, using a fall prediction machine learning model that is configured to process a user feature data object, a fall prediction data object, wherein: the fall prediction machine learning model is generated based at least in part on optimizing a custom loss model, the custom loss model comprises a fall likelihood component and a fall cause component, and the custom loss model is generated in accordance with a custom loss generation routine that comprises: identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications; generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications; for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object; generating the fall likelihood component based at least in part the fall likelihood loss values for the one or more training user feature data objects; and generating the fall cause component based at least in part on the fall cause loss values for the one or more training user feature data objects; and perform one or more prediction-based actions based at least in part on the fall likelihood prediction.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of a system that can be used to practice embodiments of the present invention;

FIG. 2 provides an example predictive data analysis computing entity in accordance with some embodiments discussed herein;

FIG. 3 provides an example external computing entity in accordance with some embodiments discussed herein;

FIG. 4 provides a flowchart diagram of an example process for generating a fall prediction data object in accordance with some embodiments discussed herein;

FIG. 5 provides a flowchart diagram of an example process for training a fall prediction machine learning model using a custom loss model in accordance with some embodiments discussed herein;

FIG. 6 provides a flowchart diagram of an example process for training a fall prediction machine learning model using a teacher machine learning model in accordance with some embodiments discussed herein;

FIG. 7 provides a flowchart diagram of an example process for generating a fall likelihood prediction data object in accordance with some embodiments discussed herein;

FIGS. 8-9 provide operational examples of two prediction-based actions that may be performed in accordance with some embodiments discussed herein;

FIG. 10 provides an operational example of two training user feature data objects in accordance with some embodiments discussed herein;

FIG. 11 provides an operational example of generating a fall likelihood component of a custom loss model in accordance with some embodiments discussed herein; and

FIGS. 12-13 provide operational example of generating fall cause loss values of the fall cause component of a custom loss model in accordance with some embodiments discussed herein.

### DETAILED DESCRIPTION

Various embodiments of the present invention are described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Advantages

Various embodiments of the present invention address technical challenges related to accurately and dynamically predicting a probability for a fall and a likely fall cause for the fall prior to the event occurring using collected data from disparate data sources. According to the Center for Disease Control and Prevention, falls are among the most common injuries among Americans above the age of 65. In the United States, one out of every four adults above the age of 65 have experienced a fall that is preventable. The risk of a user's fall risk may be associated with a plurality of factors including but not limited to intrinsic factors specific to the user, such as the user's age, gender, fall history, medical conditions, prescription drug regimen, fall history, cause for a previous fall and overall mobility, as well as extrinsic factors, such as the user's footwear and environmental elements like uneven flooring or loose rugs. While these factors are known to contribute to a user's fall risk, no current methodology exists capable of leveraging information associated with a plurality of the aforementioned risk factors into a single methodology. Furthermore, current methodologies configured to mitigate fall risk are not capable of determining a likely fall cause. Therefore, while such methodologies may be able to predict a fall, these methodologies fall short as they are unable to identify a likely fall cause and thus, the user is unable to take preventative actions to reduce his/her fall likelihood.

To address the above-noted technical challenges associated with accurately and dynamically predicting a probability for a fall and a likely cause for the fall prior to the event occurring, various embodiments of the present invention describe a fall prediction machine learning model that is configured to process one or more user feature data objects to generate a fall prediction data object describing a fall likelihood prediction, and in an instance when the fall likelihood prediction is an affirmative likelihood prediction, one or more fall cause predictions and/or a fall timing prediction. The user feature data object may comprise one or more numerical timeseries feature data fields, one or more categorical timeseries feature data fields, and one or more static feature data fields such that data from disparate data sources may be used as input for the fall prediction machine learning model. Further, in the event the fall likelihood prediction is an affirmative likelihood prediction, a fall prediction notification describing the fall prediction data object may be sent to an edge client computing entity such that an end user may be notified of a potential fall prior to the event occurring.

Additionally, the fall prediction machine learning model may be trained based at least in part on distillation loss, which is a combination of custom loss generated by a custom loss model and Kullback-Leibler (KL) divergence. Use of a distillation loss allows for the fall prediction machine learning model to process fewer parameters as compared to a trained teacher fall prediction machine learning model. As such, the fall prediction machine learning model may generate a fall prediction data object describing a fall likelihood prediction and one or more fall cause predictions while reducing the computational complexity of the runtime operations and thus, resulting in a more time efficient and less computationally resource-intensive method to generate a fall prediction data object for a user.

In some embodiments, to address the technical challenges associated with accurately and dynamically predicting a probability for a fall and a likely cause for the fall prior to the event occurring using collected data from disparate data sources, various embodiments of the present invention describe a fall prediction machine learning model capable of receiving input from disparate data sources and generating a fall prediction data object, indicative of a predicted fall probability and a likely cause for a fall. The fall prediction machine learning model may be trained based at least in part on a distillation loss, which is a combination of custom loss generated by a custom loss model and KL divergence. The use of a distillation loss, allows for the fall prediction machine learning model to process fewer parameters, as compared to a trained teacher fall prediction machine learning model. This in turn improves the computational efficiency of computer-implemented modules that perform operations corresponding to the fall prediction machine learning and/or enables performing operations of such modules using less resource-ready edge computing platforms. As such, the fall prediction machine learning model may generate an accurate fall prediction data object describing a fall likelihood prediction and one or more fall cause predictions while reducing the computational complexity of the runtime operations, thus resulting in a more time efficient and less computationally resource-intensive method to generate a fall prediction data object for a user.

### II. Definitions of Certain Terms

The term “user feature data object” may refer to an electronically-stored data construct that is configured to describe data describing features/activities of a user that is collected from one or more data sources. As will be recognized, a user feature data object may be represented as one or more vectors, embeddings, datasets, and/or the like. In some embodiments, the collected data may describe the user's speed of motion, orientation, medication intake, blood glucose levels, food and/or fluid intake, age, gender, medical history, activities, conditions, ambient conditions such as weather conditions, lighting conditions, and environmental surroundings, as well as any other information pertaining to the user within a predetermined time window. In some embodiments, each collected data item associated with a user may be associated with a timestamp. In some embodiments, the predetermined time window may configurable by a user. The collected data may be collected by any suitable device, such as an accelerometer, gyroscope, biometric sensors, mobile devices, light sensors, temperature sensors, pressure sensors, computing entities, or any other device capable of transmitting user data for processing. In some embodiments, the user feature data object may comprise one or more numerical timeseries feature data fields, one or more categorical timeseries feature data fields, and one or more static feature data fields. In some embodiments, the one or more numerical timeseries data fields may be processed to remove outliers such that the one or more numerical timeseries data fields are normalized to have zero mean and unit variance.

The term “training user feature data object” may refer to an electronically-stored data construct that is configured to describe a user feature data object that is associated with a ground-truth fall prediction (e.g., a ground-truth fall prediction that describes whether the user feature data object is associated with a recorded user fall, and if the user feature data object is associated with a recorded user fall, one or more recorded causes of the recorded user fall). As will be recognized, a training user feature data object may be represented as one or more vectors, embeddings, datasets, and/or the like. The input data corresponding to the one or more training user feature data objects may describe a user's speed of motion, orientation, medication intake, blood glucose levels, food and/or fluid intake, age, gender, medical history, ambient conditions such as weather conditions, lighting conditions, and environmental surroundings, as well as any other information pertaining to the user within a predetermined time window. The collected data may be collected by any suitable device, such as an accelerometer, gyroscope, biometric sensors, mobile devices, light sensors, temperature sensors, pressure sensors, computing entities, or any other device capable of transmitting user data for processing. In some embodiments, the training user feature data object may comprise one or more numerical timeseries feature data fields, one or more categorical timeseries feature data fields, and one or more static feature data fields. In some embodiments, each ground-truth fall prediction may describe a ground-truth fall likelihood prediction and one or more ground-truth fall cause indications. The ground truth fall likelihood prediction may be indicative of whether a user experienced a fall and the one or more ground-truth fall cause indications may be indicative of whether the fall was caused by one or more cause indications describing one or more candidate/plausible causes for a user fall. In some embodiments, the training user feature data object may be used by a custom loss model and/or a distillation loss model to train a fall prediction machine learning model.

The term “fall prediction machine learning model” may refer to an electronically-stored data construct that is configured to describe parameters, hyper-parameters, and/or stored operations of a machine learning model that is configured to process a user feature data object in order to generate a fall prediction data object with respect to a user described by the user feature data object. In some embodiments, the fall prediction data object may comprise a fall likelihood prediction indicative of the probability a user may fall, and in an instance where the fall likelihood prediction is an affirmative likelihood prediction, one or more fall cause predictions indicative of a likely cause for the user's predicted fall. In some embodiments, the fall prediction data object may further comprise a fall timing prediction indicative of a time range in which the user may fall. In some embodiments, the fall prediction machine learning model is a machine learning model comprising a first recurrent neural network (RNN) framework, a second RNN framework, a fully connected neural network framework, and an ensemble machine learning framework. The first RNN may be configured to process the one or more numerical timeseries feature data fields described by the user feature data object to generate a numerical timeseries embedding for the user feature data object. The second RNN framework may be configured to generate a categorical timeseries embedding for the user feature data object. The fully connected neural network frame may be configured to process the one or more static feature data fields to generate a static embedding for the user feature data object. The ensemble machine learning framework may be configured to generate the fall likelihood prediction based at least in part on the numerical timeseries embedding, the categorical timeseries embedding, and the static embedding. In some embodiments, the fall prediction machine learning model may be trained based at least in part on a distillation loss, which is a combination of custom loss and KL divergence. In some embodiments, the parameters and/or hyper-parameters of a fall prediction machine learning model may be represented as values in a two-dimensional array, such as a matrix. In some embodiments, subsequent to training, parameters of a fall prediction machine learning model are quantized (e.g., using TF Lite quantization models).

The term “custom loss model” may refer to an electronically-stored data construct that is configured to describe parameters, hyper-parameters, and/or stored operations of a model that is configured to process one or more training user feature data objects to generate a fall likelihood component and a fall cause component. The custom loss model may be configured to generate one or more inferred fall likelihood predictions for the one or more training user feature data objects using the fall prediction machine learning model and generate a fall likelihood loss value and one or more fall cause loss values. The fall likelihood loss value may be based at least in part on the ground truth fall likelihood prediction for the training user feature data object and the inferred fall likelihood prediction for the training user feature data object. The one or more fall cause loss values may be based at least in part on the one or more ground-truth fall cause indications and the one or more inferred fall cause indications for the training user feature data object. In some embodiments, the custom loss model may be configured to generate a fall likelihood component based at least in part on each fall likelihood loss value and a fall cause component based at least in part on each of the one or more fall cause loss values.

The term “trained teacher fall prediction machine learning model” ”may refer to an electronically-stored data construct that is configured to describe parameters, hyper-parameters, and/or stored operations of a machine learning model that is configured to process a user feature data object in order to generate one or more teacher outputs. In some embodiments, the trained teacher fall prediction machine learning model is trained using a custom loss model. In some embodiments, the trained teacher fall prediction machine learning model is a machine learning model comprising a first recurrent neural network (RNN) framework, a second RNN framework, a fully connected neural network framework, and an ensemble machine learning framework. The first RNN may be configured to process the one or more numerical timeseries feature data fields described by the user feature data object to generate a numerical timeseries embedding for the user feature data object. The second RNN framework may be configured to generate a categorical timeseries embedding for the user feature data object. The fully connected neural network frame may be configured to process the one or more static feature data fields to generate a static embedding for the user feature data object. The ensemble machine learning framework may be configured to generate one or more teacher outputs based at least in part on the numerical timeseries embedding, the categorical timeseries embedding, and the static embedding. In some embodiments, the one or more teacher outputs of the trained teacher fall prediction machine learning model may be used in a distillation loss to train the fall prediction machine learning model. In some embodiments, the parameters and/or hyper-parameters of a fall prediction machine learning model may be represented as values in a two-dimensional array, such as a matrix.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatuses, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Framework

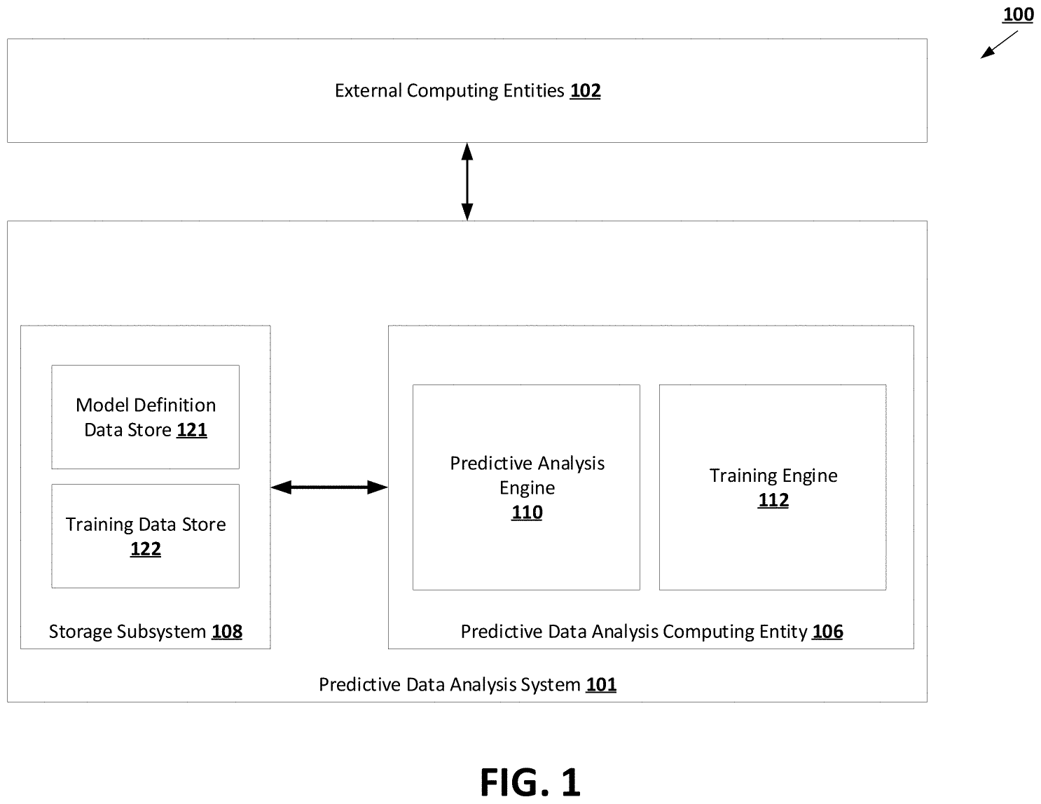

FIG. 1 is a schematic diagram of an example system architecture 100 for performing predictive data analysis operations and for performing one or more prediction-based actions (e.g., generating corresponding user interface data). The system architecture 100 includes a predictive data analysis system 101 comprising a predictive data analysis computing entity 106 configured to generate predictive outputs that can be used to perform one or more prediction-based actions. The predictive data analysis system 101 may communicate with one or more external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like). An example of a prediction that may be generated by using the system architecture 100 is to a generate predicted disease score associated with a target user depicted in a video stream data object.

The system architecture 100 includes a storage subsystem 108 configured to store at least a portion of the data utilized by the predictive data analysis system 101. The predictive data analysis computing entity 106 may be in communication with one or more external computing entities 102. The predictive data analysis computing entity 106 may be configured to train a prediction model based at least in part on the training data store 122 stored in the storage subsystem 108, store trained prediction models as part of the model definition data store 121 stored in the storage subsystem 108, utilize trained models to generate predictions based at least in part on prediction inputs provided by an external computing entity 102, and perform prediction-based actions based at least in part on the generated predictions. The storage subsystem may be configured to store the model definition data store 121 for one or more predictive analysis models and the training data store 122 uses to train one or more predictive analysis models. The predictive data analysis computing entity 106 may be configured to receive requests and/or data from external computing entities 102, process the requests and/or data to generate predictive outputs (e.g., predictive data analysis data objects), and provide the predictive outputs to the external computing entities 102. The external computing entity 102 (e.g., management computing entity) may periodically update/provide raw input data (e.g., data objects describing primary events and/or secondary events) to the predictive data analysis system 101. The external computing entities 102 may further generate user interface data (e.g., one or more data objects) corresponding to the predictive outputs and may provide (e.g., transmit, send and/or the like) the user interface data corresponding with the predictive outputs for presentation to user computing entities operated by end-users.

The storage subsystem 108 may be configured to store at least a portion of the data utilized by the predictive data analysis computing entity 106 to perform predictive data analysis steps/operations and tasks. The storage subsystem 108 may be configured to store at least a portion of operational data and/or operational configuration data including operational instructions and parameters utilized by the predictive data analysis computing entity 106 to perform predictive data analysis steps/operations in response to requests. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The predictive data analysis computing entity 106 includes a predictive analysis engine 110 and a training engine 112. The predictive analysis engine 110 may be configured to perform predictive data analysis based at least in part on a received user feature data object. For example, the predictive analysis engine 110 may be configured to one or more prediction based actions based at least in part on a fall likelihood prediction. The training engine 112 may be configured to train the predictive analysis engine 110 in accordance with the training data store 122 stored in the storage subsystem 108.

### Exemplary Predictive Data Analysis Computing Entity

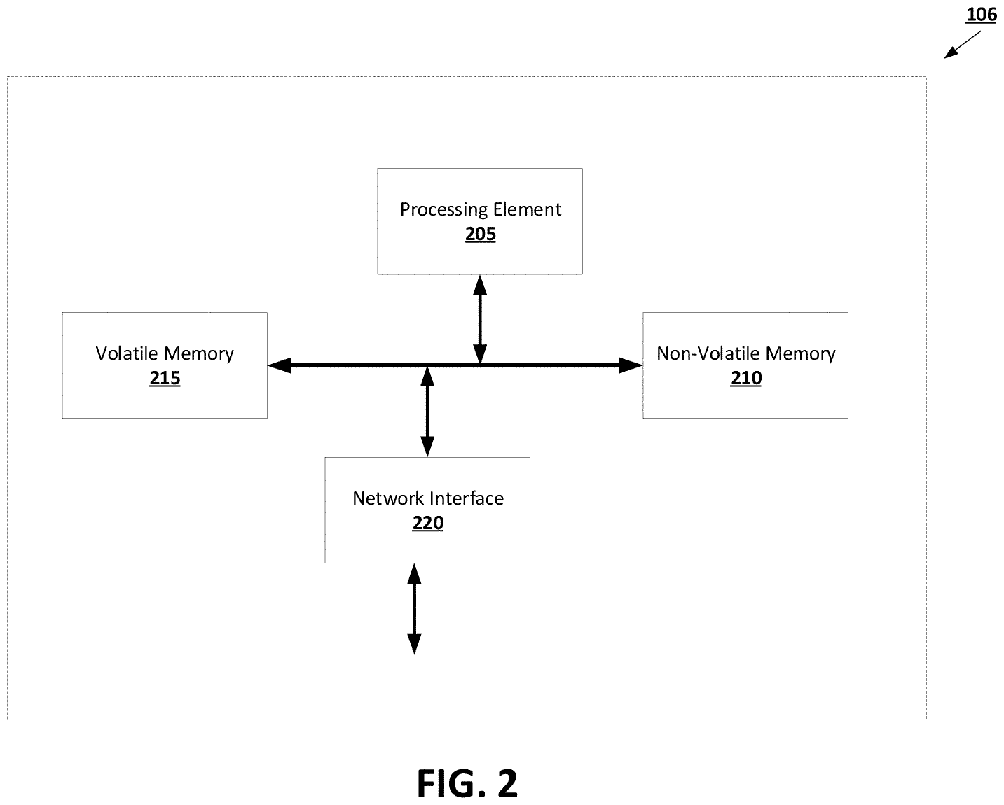

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

As indicated, in one embodiment, the predictive data analysis computing entity 106 may also include a network interface 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include or be in communication with a processing element 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include or be in communication with non-volatile media (also referred to as non-volatile storage, memory, memory storage, memory circuitry and/or similar terms used herein interchangeably). In one embodiment, the non-volatile storage or memory may include at least one non-volatile memory 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 may further include or be in communication with volatile media (also referred to as volatile storage, memory, memory storage, memory circuitry and/or similar terms used herein interchangeably). In one embodiment, the volatile storage or memory may also include at least one volatile memory 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include a network interface 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include or be in communication with one or more input elements, such as a keyboard input, a mouse input, a touch screen/display input, motion input, movement input, audio input, pointing device input, joystick input, keypad input, and/or the like. The predictive data analysis computing entity 106 may also include or be in communication with one or more output elements (not shown), such as audio output, video output, screen/display output, motion output, movement output, and/or the like.

### Exemplary External Computing Entity

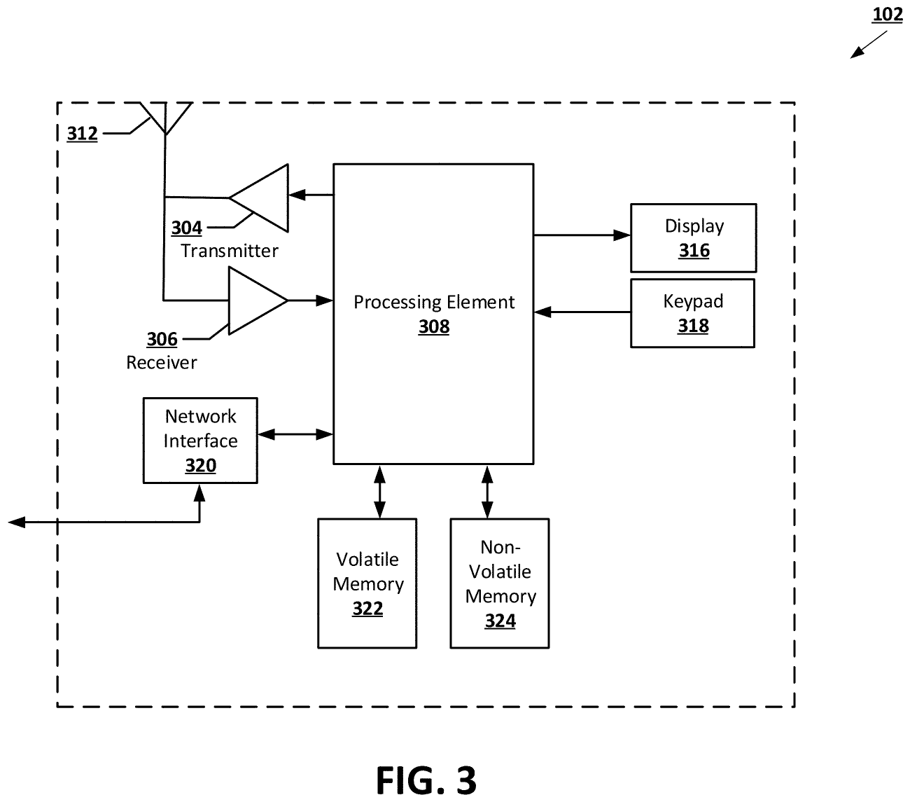

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (US SD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive data analysis computing entity 106 and/or various other computing entities.

In another embodiment, the external computing entity 102 may include one or more components or functionality that are the same or similar to those of the predictive data analysis computing entity 106, as described in greater detail above. As will be recognized, these frameworks and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a video capture device (e.g., camera), a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

A user's associated fall risk is a linked to a multitude of factors such that an accurate fall risk assessment requires processing of data from disparate data sources. However, current methodologies configured to predict a user's fall risk are limited, as these methodologies are unable to process data from disparate data sources to generate a dynamic fall prediction for the user. For example, consideration of only the medical history of a user may not predict a fall caused by the user forgetting to take his/her prescribed medication. Additionally, data from disparate data sources may contain data with different attribute types, such as numeric, categorical, and/or status attribute types, thus further complicating consideration of data from disparate data sources.

Furthermore, current methodologies are unable to dynamically predict a fall cause, even if a fall is predicted before the event. Therefore, current methodologies are unable to advise a user on corrective actions he/she may take to reduce his/her fall likelihood, such as taking his/her prescribed medication.

As such, to address the technical challenges associated with accurately and dynamically predicting a probability for a fall and a likely cause for the fall prior to the event occurring using collected data from disparate data sources, various embodiments of the present invention describe a fall prediction machine learning model capable of receiving input from disparate data sources and generating a fall prediction data object, indicative of a predicted fall probability and a likely cause for a fall. The fall prediction machine learning model may be trained based at least in part on distillation loss, which is a combination of custom loss generated by a custom loss model and KL divergence. The use of a custom loss and a distillation loss allows for the fall prediction machine learning model to process fewer parameters, as compared to a trained teacher fall prediction machine learning model, which in turn improves the computational efficiency of computer-implemented modules that perform operations corresponding to the fall prediction machine learning and/or enables performing operations of such modules using less resource-ready edge computing platforms. As such, the fall prediction machine learning model may generate an accurate fall prediction data object describing a fall likelihood prediction and one or more fall cause predictions while reducing the computational complexity of the runtime operations, thus resulting in a more time efficient and less computationally resource-intensive method to generate a fall prediction data object for a user.

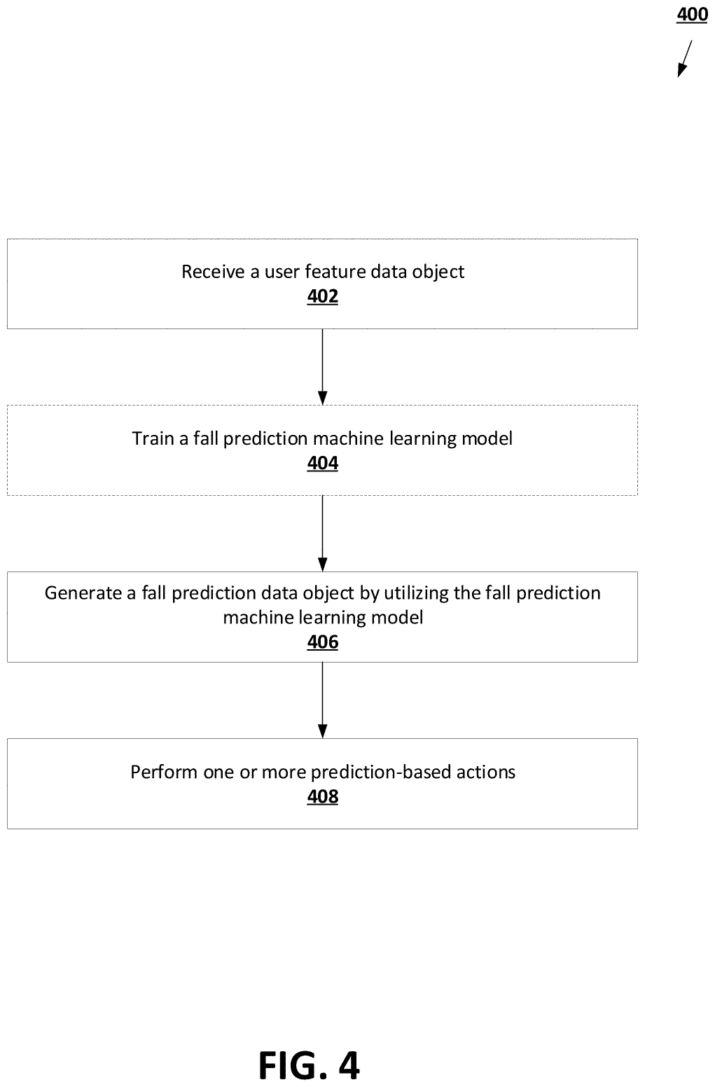

FIG. 4 is a flowchart diagram of an example process 400 for generating a fall prediction data object for a user. Via the various steps/operations of the process 400, the predictive data analysis computing entity 106 can accurately and dynamically generate a real-time fall prediction data object for a user described by a user feature data object.

The process 400 begins at step/operation 402 when the predictive analysis engine 110 of the predictive data analysis computing entity 106 receives a user feature data object indicative of data pertaining to a user. For example, the user feature data object may describe the associated user's speed of motion, orientation, medication intake, blood glucose levels, food and/or fluid intake, age, gender, medical history, fall history, one or more causes for one or more previous falls and the like. The user feature data object may also describe the current time of day, week, or year for each collected data item, as well as in some embodiments the user's environmental surroundings, and the like.

In some embodiments, the user feature data object comprises user data from one or more data sources. For example, the user feature data object may describe data collected from an accelerometer, gyroscope, biometric sensors, mobile devices, light sensors, temperature sensors, pressure sensors, computing entities, or any other device capable of transmitting user data. In this way, the user may leverage existing devices he/she already routinely uses or may incorporate new devices to describe additional data fields and improve the robustness of the collected user data for the user feature data object.

In some embodiments, the user data from one or more data sources may be pre-processed by a pre-processing layer. In some embodiments, the pre-processing layer may process the one or more numerical timeseries data fields to remove outliers such that the one or more numerical timeseries data fields are normalized to have zero mean and unit variance. In some embodiments, the pre-processed user data from one or more data sources may additionally be processed by a feature engineering layer. The feature engineering layer may extract one or more data fields from the pre-processed user data to generate the one or more data fields of the user feature data object.

In some embodiments, the user feature data object may comprise data with various data attribute types. For example, the user feature data object may comprise one or more numerical timeseries feature data fields, one or more categorical timeseries feature data fields, and one or more static feature data fields. Numerical timeseries feature data fields may include data fields associated with dynamic numerical data, such as a sequence of accelerometer coordinates, a sequence of gyroscope coordinates, a sequence of temperature, a sequence of distance from a proximity sensor, and the like. Categorical timeseries feature data fields may include data fields associated with dynamic categorical data, such as a sequence of medication intake (e.g., national drug codes (NDC)), a sequence of medical history codes (e.g., international classification of disease codes (ICD)), and the like. Static feature data fields may include static data fields, such as age, gender, and the like that do not change over time.

Optionally, at step/operation 404, the training engine 112 of the predictive data analysis computing entity 106 may train a fall prediction machine learning model. The training engine 112 may access a plurality of training user feature data objects, for example, from training data store 122. Using the plurality of training user feature data objects, the training engine 112 may train a fall prediction machine learning model to generate a fall prediction data object. In some embodiments, the training engine 112 may train a fall prediction machine learning model based at least in part on a custom loss generated by a custom loss model. In some embodiments, the training engine 112 may train the fall prediction machine learning model based at least in part on a distillation loss.

In some embodiments, the training engine 112 may train a fall prediction machine learning model (or other machine learning model, such as a teacher machine learning model, as described below) using a custom loss model. In some embodiments, the custom loss model is characterized by an overall fall loss component (as described below) that is determined using the below equation:

$$Overall Loss = {\sum }_{i = 0}^{n} loss\_per {\_observation}_{i} \qquad Equation 1$$

In some embodiments, loss_per_observationᵢ is determined using the equation loss_per_observationᵢ=loss_fallᵢ+circumstantial_lossᵢ, where the loss_fallᵢ is the fall likelihood component for the observation i as described below and circumstantial_lossᵢ is the fall cause component for the observation i as described below. In some embodiments, loss_fallᵢ is determined using the equation loss_fallᵢ=−(y_fallᵢ*log(p_fallᵢ)+[(1−y_fallᵢ)*log(1−p_fallᵢ)], where y_fallᵢ is the ground-truth fall likelihood indication for the observation i and y_fallᵢ is the inferred fall likelihood prediction for the observation for the observation i. In some embodiments, given a set of n cause indications, the circumstantial_lossᵢ=loss_cause c1ᵢ+ . . . +loss_cause_c1ₙ, where loss cause cm, is the fall cause loss value for an mth cause of the n cause indications in relation to the observation i. In some embodiments, loss cause cm, is the determined using the below equation:

$$loss\_cause {\_cm}_{i} = \{ \begin{matrix}\begin{matrix}- [ ( y\_cause {\_cm}_{i} * log ( p\_cause {\_cm}_{i} ) ) + \\ ( ( 1 - y\_cause {\_cm}_{i} ) * log ( 1 - p\_cause {\_cm}_{i} ) ) ,\end{matrix} & if {y\_fall}_{i} = 1 \\ 0 , & if {y\_fall}_{i} = 0\end{matrix} \qquad Equation 2$$

In Equation 2, y_cause_cmᵢ is the ground-truth fall cause indication for the mth cause of the n cause indications in relation to the observation i, p_cause_cmᵢ is the inferred fall cause prediction for the mth cause of the n cause indications in relation to the observation i, and y_fallᵢ is the ground-truth fall likelihood indication for the observation i.

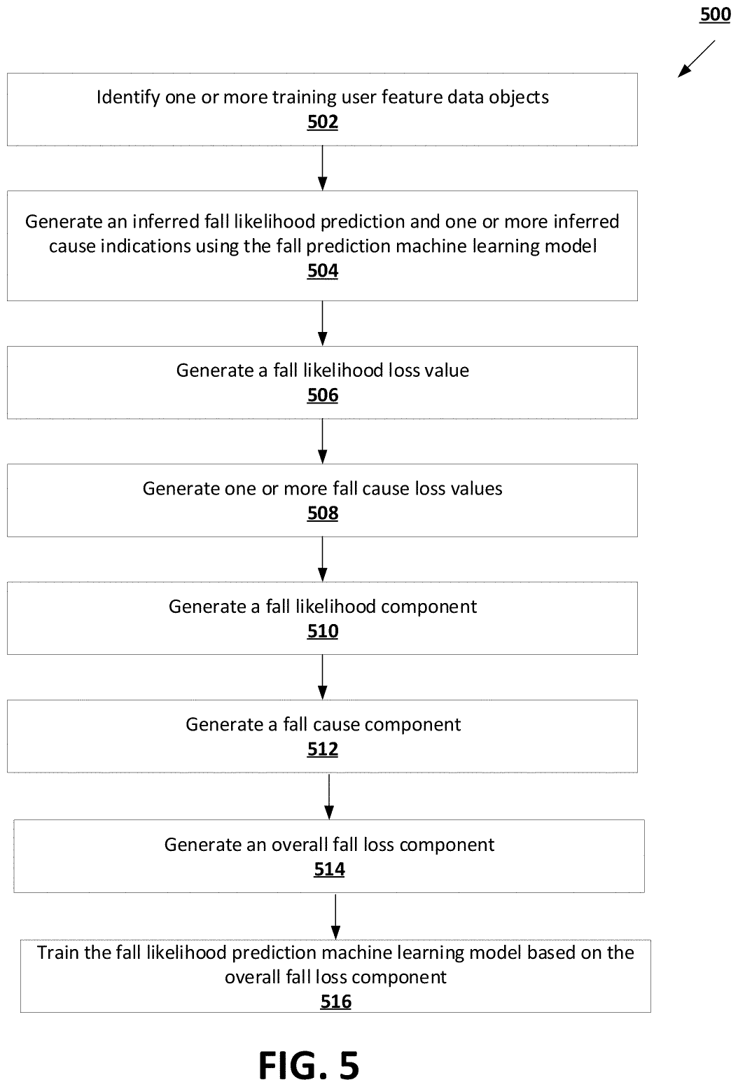

In some embodiments, step/operation 404 may be performed in accordance with the various steps/operations of the process 500 depicted in FIG. 5, which is a flowchart diagram of an example process for training one or more fall prediction machine learning models based at least in part on a custom loss model.

The process 500 begins at step/operation 502, when the training engine 112 identifies one or more training user feature data objects by utilizing a custom loss model in accordance with a custom loss generation routine. The one or more training user feature data objects may be identified from, for example, a training data store 122. In some embodiments, the training engine 112 receives the one or more training user feature data objects from the training data store 122. In some embodiments, training engine 112 may periodically (e.g., weekly, monthly, or bi-annually) receive one or more training user feature data objects. In this way, a fall prediction machine learning model may be able maintain an updated fall prediction machine learning model to facilitate generation of an accurate fall prediction data object for a user.

The one or more training user feature data objects may describe collected data pertaining to one or more users. The one or more training user feature data objects may describe a user's speed of motion, orientation, medication intake, blood glucose levels, food and/or fluid intake, age, gender, medical history, ambient conditions such as weather conditions, lighting conditions, and environmental surroundings, as well as any other information pertaining to the user that is obtained/recorded within a predetermined time window. In some embodiments, the predetermined time window may configurable by a user. For example, the predetermined time window may be 24 hours, such that data collected within 24 hours is processed. The collected data may be collected by any suitable device, such as an accelerometer, gyroscope, biometric sensors, mobile devices, light sensors, temperature sensors, pressure sensors, computing entities, or any other device capable of transmitting user data for processing. In some embodiments, the training user feature data object may comprise one or more numerical timeseries feature data fields, one or more categorical timeseries feature data fields, and one or more static feature data fields.

Each of the one or more training user feature data objects may be associated with one or more ground-truth fall predictions. In some embodiments, the ground-truth fall prediction may describe a ground-truth fall likelihood prediction and one or more ground-truth fall cause indications. The ground-truth fall likelihood prediction may be associated with a binary value indicative of whether a user experienced a fall. For example, a ground-truth likelihood prediction value of 0 may be indicative that the user did not experience a fall and a ground-truth likelihood prediction value of 1 may be indicative that the user experienced a fall. The one or more ground-truth fall cause indications may each be associated with a binary value indicative of whether the fall was caused by the particular fall cause indication. For example, the one or more ground-truth fall cause indications may include a drop in glucose, pre-existing condition, or drop in blood pressure. If a fall was caused by a drop in glucose, the ground-truth fall cause indication value corresponding to a drop in glucose may be 1 and the other ground-truth fall cause indication values may be 0. The one or more ground-truth fall cause indications may indicate more than one likely cause for a fall.

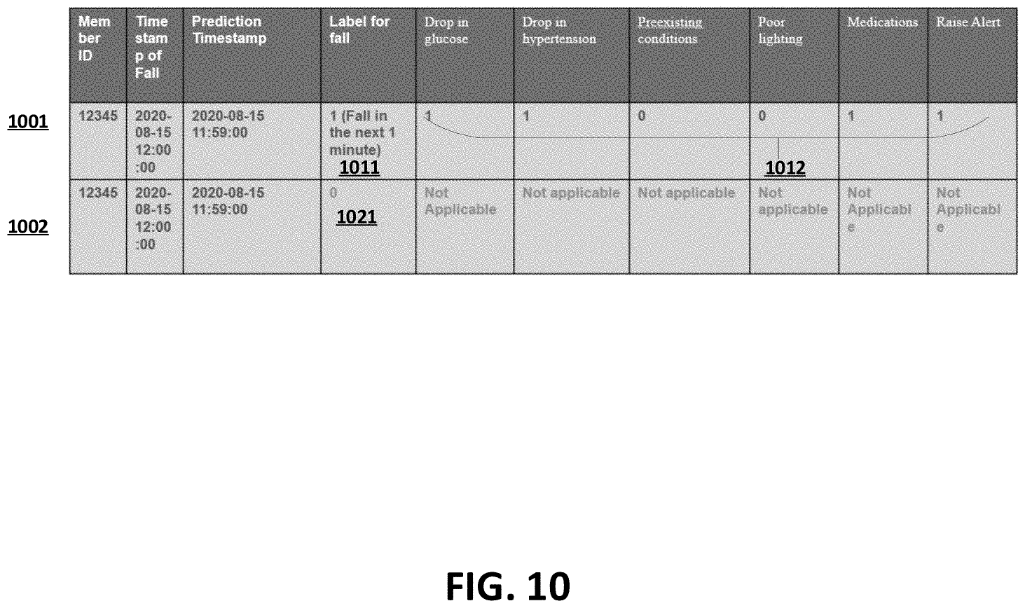

An operational example of two training user feature data objects is depicted in FIG. 10. As depicted in FIG. 10, because the training user feature data object 1001 is associated with an affirmative ground-truth fall likelihood indication 1011, it is associated with the ground-truth fall cause indications 1012, while the training user feature data object 1002 is not associated with any ground-truth fall cause indications because the training user feature data object 1002 is associated with a negative ground-truth fall likelihood indication 1021.

At step/operation 504, the training engine 112 may generate one or more inferred fall predictions for the one or more training user feature data objects by utilizing the custom loss model. The custom loss model may generate the one or more inferred fall predictions by utilizing the fall prediction machine learning model. The inferred fall prediction may describe an inferred fall likelihood prediction and one or more fall cause indications. The inferred fall likelihood prediction may be indicative of a probability that a user, associated with a particular training user feature data object, will experience a fall, as predicted by the fall prediction machine learning model. In some embodiments, the inferred fall likelihood prediction may be associated with a numerical value between 0 and 1. The one or more inferred fall cause indications may be indicative of a probability that one or more inferred fall cause indications are responsible for a user fall, as predicted by the fall predication machine learning model. In some embodiments, the one or more inferred fall cause indications may be associated with a numerical value between 0 and 1. In some embodiments, the one or more inferred fall cause indications may correspond to the one or more ground-truth fall cause indications, such that the one or more inferred fall cause indications match the one or more ground-truth fall cause indications.

At step/operation 506, training engine 112 may generate a fall likelihood loss value for each training user feature data object of the one or more user feature data objects by utilizing the custom loss model. The custom loss model may generate the fall likelihood loss value based at least in part on the ground-truth fall likelihood prediction for a training user feature data object and the inferred fall likelihood prediction for the training user feature data object. The fall likelihood loss value may be indicative of the accuracy of a set of fall likelihood predictions by the fall prediction machine learning model as determined based at least in part on a set of ground-truth fall likelihood indications for the set of fall likelihood prediction. For example, the custom loss model may generate a fall likelihood loss value, wherein the closer the fall likelihood loss value is to 0, the more accurately the fall prediction machine learning model predicted whether a user would experience a fall as determined based at least in part on a set of ground-truth fall likelihood indications for the set of fall likelihood prediction.

At step/operation 508, training engine 112 may generate one or more fall cause loss values for each training user feature data object by utilizing the custom loss model. In some embodiments, the generation of the one or more fall cause loss values may occur before, after, or simultaneously with the generation of a fall likelihood loss value as described in step/operation 506. The custom loss model may generate the one or more fall cause loss values based at least in part on the one or more ground-truth fall cause indications for the training user feature data object and the one or more inferred fall cause indications for the user feature data object. The one or more fall cause loss values may be indicative of the accuracy of a set of inferred cause indications generated by the fall prediction machine learning model as determined based at least in part on a set of ground-truth fall cause indications corresponding to the one or more fall cause loss values. The one or more fall cause loss values may in some embodiments be indicative of both how accurate the fall prediction machine learning model is at correctly predicting a fall cause as well as how accurate the fall prediction machine learning model is at correctly not predicting a fall cause. For example, if the one or more ground-truth fall cause indications were indicative that a user fall was caused by a drop in glucose, the ground-truth fall cause indication value corresponding to a drop in glucose may be 1 while the ground-truth fall cause indication value corresponding to a drop in blood pressure value may be 0. In some embodiments, in the exemplary scenario described above, a fall prediction machine learning model may generate one or more inferred fall cause indications indicative of a value of 0.8 associated with an inferred fall cause indication corresponding to a drop in glucose and a value of 0.1 associated with the inferred fall cause indications corresponding to a drop in blood pressure. In this way, the fall prediction machine learning model accurately predicted that a fall was likely caused by a drop in a user's glucose but was not caused by a drop in blood pressure.

The custom loss model may generate one or more fall cause loss values, wherein the closer a fall cause loss value is to 0, the more accurately the fall prediction machine learning model predicted the cause for a fall. In some embodiments, the one or more fall cause loss values may correspond to an individual ground-truth fall cause indication and/or inferred fall cause indication. In some embodiments, if the training user feature data object of the one or more training user feature data objects is not associated with a fall, a fall cause loss value of 0 is automatically generated for training user feature data object.

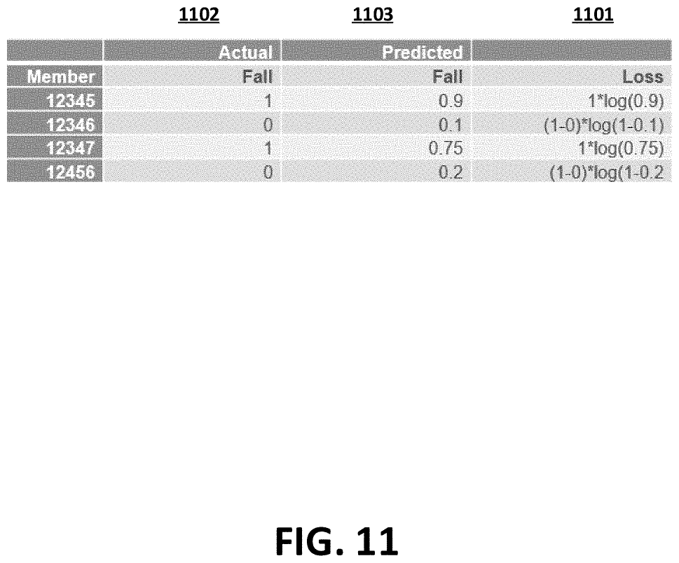

At step/operation 510, training engine 112 may generate a fall likelihood component by utilizing the custom loss model. In some embodiments, the custom loss model may generate the fall likelihood component based at least in part on the fall likelihood loss value for the one or more training user feature data objects. In some embodiments, the custom loss model may generate the fall likelihood component by summing the one or more fall likelihood loss values associated each of the training user feature data objects in the one or more user feature data objects. For example, if four training user feature data objects are processed by the custom loss model, each of the four training user feature data objects may be associated with a fall likelihood loss value based as described by step/operation 506, such as fall likelihood loss values of 0.1, 0.1, 0.3, and 0.2. The fall likelihood component may be generated by the custom loss model by summing each of the one or more fall likelihood loss values, such that the value of the fall likelihood component may be 0.7. The fall likelihood component may be indicative of the accuracy of the fall prediction machine learning model with regard to predicting whether a user will experience a fall. The closer the fall likelihood component value is to 0, the more accurate the fall prediction machine learning model is at predicting a fall likelihood prediction. In some embodiments, the custom loss model may train the fall prediction machine learning model based at least in part on the fall likelihood component. FIG. 11 depicts an operational example of generating sub-components 1101 of a fall likelihood component of a custom loss model using the ground-truth fall likelihood indications 1102 and the predicted fall likelihood predictions 1103.

At step/operation 512, training engine 112 may generate a fall cause component by utilizing the custom loss model. In some embodiments, the custom loss model may generate the fall loss component based at least in part on the one or more fall cause loss values for the one or more training user feature data object. In some embodiments, the custom loss model may generate the fall loss component by summing the one or more fall cause loss values each corresponding to one or more of the ground-truth fall cause indications and/or the one or more inferred fall cause indications for the one or more training user feature data objects. For example, if two training user feature data objects are processed by the custom loss model, each of the four training user feature data objects may be associated with one or more fall cause loss values for one or more ground-truth fall cause indications and/or the one or more inferred fall cause indications. The one or more ground-truth fall cause indications and/or the one or more inferred fall cause indications may include a drop in glucose, a pre-existing condition, or a drop in blood pressure. In this exemplary scenario, the first training feature data object may correspond to one or more fall cause loss values of 0.1, 0.2, and 0.1 for a drop in glucose, a pre-existing condition, or a drop in blood pressure, respectively. The second training feature data object may correspond to one or more fall cause loss values of 0.2, 0.1, and 0.2 for a drop in glucose, a pre-existing condition, or a drop in blood pressure, respectively. The fall cause component may be generated by the custom loss model by summing each of the one or more fall cause loss values, such that the value of the fall cause component may be 0.9. The fall cause component may be indicative of the accuracy of the fall prediction machine learning model with regard to predicting a fall cause for a user in the event of a fall. The closer the fall cause component value is to 0, the more accurate the fall prediction machine learning model is at predicting one or more fall cause indications. In some embodiments, the custom loss model may train the fall prediction machine learning model based at least in part on the fall cause component.

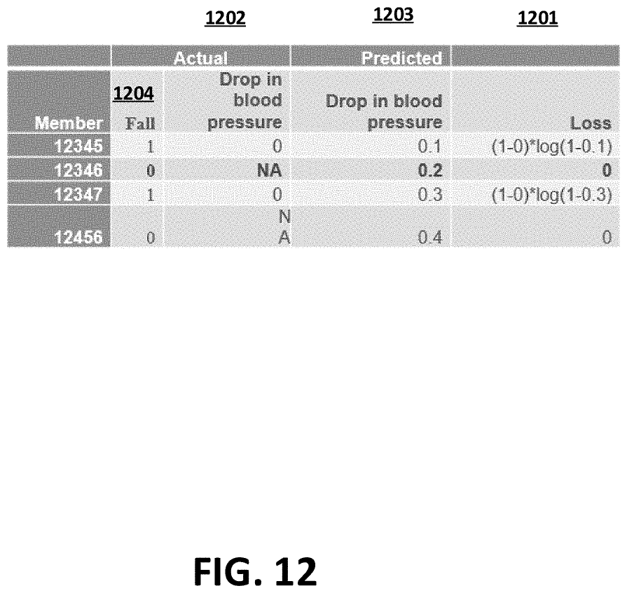

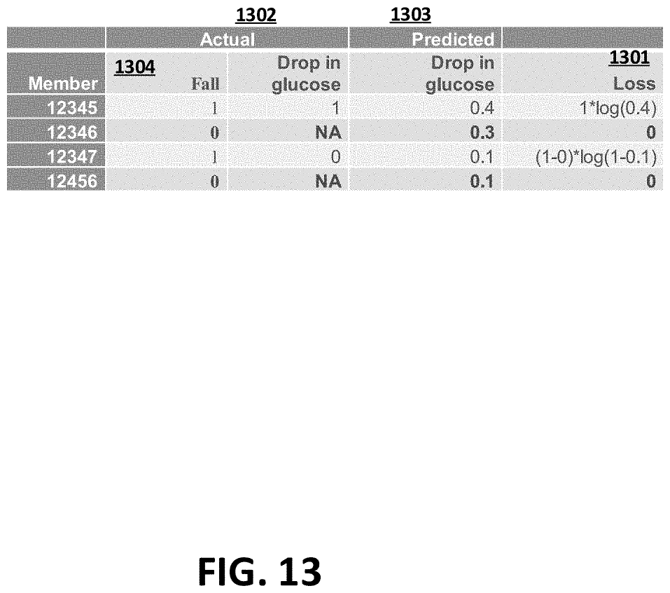

FIG. 12 depicts an operational example of generating sub-components 1201 of a fall likelihood cause of a custom loss model for a cause related to a drop in blood pressure using the ground-truth fall cause indications 1202, the predicted fall cause predictions 1203, and the ground-truth fall likelihood predictions 1204. FIG. 13 depicts an operational example of generating sub-components 1301 of a fall likelihood cause of a custom loss model for a cause related to a drop in glucose using the ground-truth fall cause indications 1302, the predicted fall cause predictions 1303, and the ground-truth fall likelihood predictions 1304.

At step/operation 514, the training engine 112 may also generate an overall fall loss component by utilizing the custom loss model. In some embodiments, the custom loss model may generate the overall fall loss component based at least in part on the fall likelihood component and the fall cause component. In some embodiments, the custom loss model may sum the fall likelihood component and the fall cause component to generate the overall fall loss component. For example, if the fall likelihood component corresponds to a value of 0.7 and the fall cause component corresponds to a value of 0.9, the overall fall loss component may correspond to a value of 1.6. The overall fall loss value may be indicative an overall accuracy of the fall prediction machine learning model as the overall fall loss value is based at least in part on the fall likelihood component and the fall cause component. In some embodiments, the custom loss model may train the fall prediction machine learning model based at least in part on the overall fall loss component.

At step/operation 516, the training engine 112 may train the fall prediction machine learning model based at least in part on the overall fall loss component. In some embodiments, by using the custom loss model, the training engine 112 may train the fall prediction machine learning model in a manner that is configured to minimize the overall fall loss component.

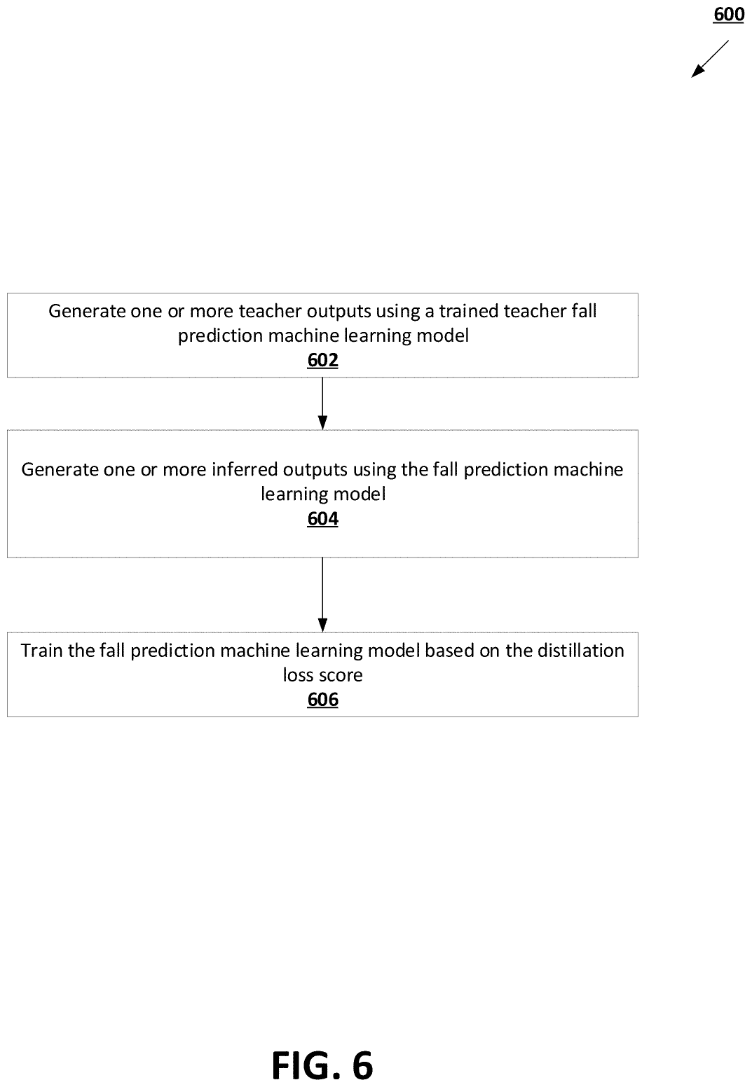

In some embodiments, step/operation 404 may also be performed in accordance with the various steps/operations of the process 600 that is depicted in FIG. 6, which is a flowchart diagram of an example process for training one or more fall prediction machine learning models based at least in part on a distillation loss.

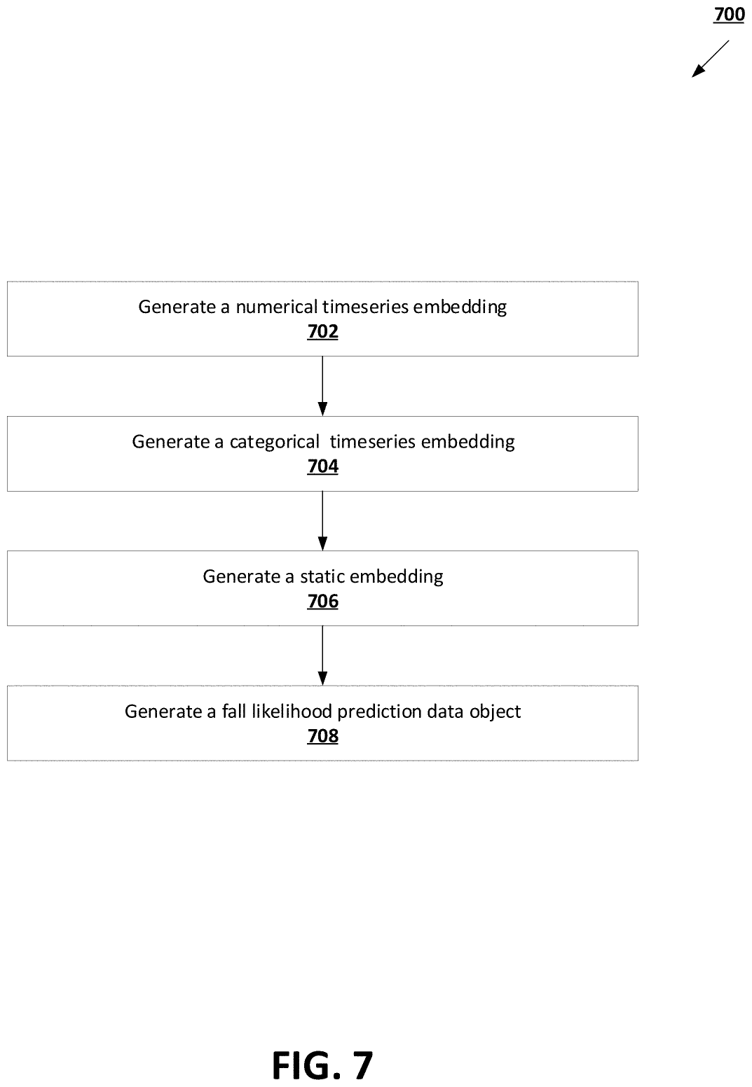

The process 600 begins at step/operation 602, when the training engine 112 generates one or more teacher outputs using a trained teacher fall prediction machine learning model. The trained teacher model may be a machine learning model configured to process one or more training user feature data objects to generate one or more teacher outputs. In some embodiments, the trained teacher fall prediction machine learning model is a machine learning model comprising a first recurrent neural network (RNN) framework, a second RNN framework, a fully connected neural network framework, and an ensemble machine learning framework. The first RNN may be configured to process the one or more numerical timeseries feature data fields described by the user feature data object to generate a numerical timeseries embedding for the user feature data object. The second RNN framework may be configured to generate a categorical timeseries embedding for the user feature data object. The fully connected neural network frame may be configured to process the one or more static feature data fields to generate a static embedding for the user feature data object. The ensemble machine learning framework may be configured to generate one or more teacher outputs based at least in part on the numerical timeseries embedding, the categorical timeseries embedding, and the static embedding. The trained teacher machine learning model may be configured to process the one or more training user feature data objects using the first RNN framework, second RNN framework, fully connected neural network framework, and ensemble machine learning framework as will be described in more detail with respect to FIG. 7. In some embodiments, at least one of the first RNN framework and the second RNN framework comprises a long short term memory (LSTM) RNN framework.

In some embodiments, the trained teacher machine learning model may have been trained using the custom loss model as previously described with respect to the process 500 in FIG. 5. In some embodiments, the one or more teacher outputs may describe a teacher fall likelihood prediction and one or more teacher fall cause indications. The teacher fall likelihood prediction and one or more teacher fall cause indications may be associated with values between 0 and 1.

At step/operation 604, the training engine 112 may generate one or more inferred outputs by using the fall prediction machine learning model. The fall prediction machine learning model may process the one or more training user feature data objects to generate one or more inferred outputs. In some embodiments, the fall prediction machine learning model may have been trained using distillation loss, which is a combination of custom loss generated by a custom loss model as previously described with respect to the process 500 in FIG. 5 and KL divergence. In some embodiments, the one or more teacher outputs may describe an inferred fall likelihood prediction and one or more inferred fall cause indications. The inferred fall likelihood prediction and one or more inferred fall likelihood predictions may be associated with values between 0 and 1. In some embodiments, the fall prediction machine learning model may be configured to process fewer parameters as compared to the trained teacher fall prediction machine learning model.

At step/operation 606, the training engine 112 may use the distillation loss to train the fall prediction machine learning model based at least in part on the distillation loss score. The distillation loss score based at least in part on the one or more teacher outputs, the one or more inferred outputs, ground truth fall likelihood and ground truth fall cause. In some embodiments, the KL divergence component of the distillation loss score may be indicative of the relative entropy between the trained teacher fall prediction machine learning model and the fall prediction machine learning model that is determined based at least in part on the one or more teacher outputs and one or more inferred outputs. In some embodiments, the relative entropy is determined based at least in part on KL divergence. The custom loss component is previously described with respect to the process 500 in FIG. 5. The fall prediction machine learning model may train the fall prediction machine learning model by minimizing the distillation loss score. In this way, the distillation loss model may use the one or more teacher outputs as generated by the trained teacher fall prediction machine learning model using more parameters, and the one or more inferred outputs as generated by the fall prediction machine learning model and ground-truth fall likelihood, to train the fall prediction machine learning model. As such, the fall prediction machine learning model may maintain accuracy while reducing the number of processed parameters, and therefore, reducing the complexity of the runtime operations.

In some embodiments, the fall prediction machine learning model is generated based at least in part on optimizing a distillation loss, which is a combination of KL divergence and custom loss generated by a custom loss model, where the custom loss model comprises a fall likelihood component and a fall cause component, and the custom loss model is generated in accordance with a custom loss generation routine that comprises: identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications; generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications; for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object; generating the fall likelihood component based at least in part the fall likelihood loss values for the one or more training user feature data objects; and generating the fall cause component based at least in part on the fall cause loss values for the one or more training user feature data objects

At step/operation 406, the predictive analysis engine 110 of predictive data analysis computing entity 106 may generate a fall prediction data object by utilizing the fall prediction machine learning model. The fall prediction data object may describe a fall likelihood prediction. The fall likelihood prediction may be indicative of whether a user is predicted to experience a fall. In some embodiments, the fall likelihood prediction is a binary value, where a fall likelihood prediction value of 1 may be indicative that a user is predicted to experience a fall and a fall likelihood prediction value of 0 may be indicative that a user is not predicted to experience a fall. In an instance where the fall likelihood prediction is affirmative, e.g., a fall is predicted, the fall prediction machine learning model may also describe one or more fall cause predictions. The one or more fall cause predictions may be a multi-class multi-label classification indicative of the one or more likely causes for a fall. For example, a fall cause prediction may be indicative that a predicted fall is likely to be caused by the user forgetting to take his/her medication. In some embodiments, in an instance where the fall likelihood prediction is affirmative, e.g., describing that a fall is predicted, the fall prediction machine learning model may also describe a fall timing prediction. The fall timing prediction may be indicative of a predictive time for a fall to occur. In some embodiments, the fall timing prediction may be indicative of an estimated time and date for a fall to occur. For example, the fall timing prediction may predict a fall to occur at 11:59:00 am on Aug. 15, 2020.

In some embodiments, step/operation 406 may also be performed in accordance with the various steps/operations of the process 700 that is depicted in FIG. 7, which is a flowchart diagram of an example process for generating a fall likelihood prediction data object. As described above, the user feature data object may comprise data with various attributes types. For example, the user feature data object may comprise one or more numerical timeseries feature data fields, one or more categorical timeseries feature data fields, and one or more static feature data fields. Numerical timeseries feature data fields may include a sequence of accelerometer coordinates, a sequence of gyroscope coordinates, a sequence of temperature, a sequence of distance from a proximity sensor, and the like. Categorical timeseries feature data fields may include a sequence of medication intake such as NDC codes, a sequence of medical history codes such as ICD codes, and the like. Static feature data fields may include age, gender, and the like. Thus, the different attribute types may be processed by the fall prediction machine learning model via various frameworks based at least in part of the attribute type. In some embodiments, the fall machine learning model may comprise a first RNN framework, a second RNN framework, a fully connected neural network framework, and an ensemble machine learning network framework.

The process 700 begins at step/operation 702, when the predictive analysis engine 110 generates one or more numerical timeseries embeddings for the user feature data object utilizing the fall prediction machine learning model. The fall prediction machine learning model may be configured to process the one or more numerical timeseries feature data fields using a first RNN framework. In some embodiments, the one or more numerical timeseries embeddings are fed as input to different branches of the first RNN framework. In some embodiments, the one or more numerical timeseries data fields may be handled for outliers such that the one or more numerical timeseries data fields are normalized to have zero mean and unit variance prior to processing by the first RNN framework.

At step/operation 704, the predictive analysis engine 110 generates one or more categorical timeseries embeddings for the user feature data object utilizing the fall prediction machine learning model. The fall prediction machine learning model may be configured to process the one or more categorical timeseries feature data fields using a second RNN framework. In some embodiments, the second RNN framework long term short memory RNN framework. In some embodiments, a vector space model process categorical timeseries feature data prior to the categorical timeseries feature data being processed by the second RNN framework. For example, if the categorical timeseries feature data describes ICD codes, the vector space model may order semantically similar ICD codes to have similar vector representations. As such, categorical timeseries feature data associated with high cardinality, such as ICD codes, may be represented as vectors such that the second RNN framework may use less computational resources to generate the one or more categorical timeseries embeddings.

At step/operation 706, the predictive analysis engine 110 generates a static embedding for the user feature data object utilizing the fall prediction machine learning model. The fall prediction machine learning model may be configured to process the one or more static feature data fields using a fully connected neural network framework. In some embodiments, the fully connected neural network framework may concatenate the last hidden state of the second RNN framework with the one or more static feature data fields into a concatenated vector. In some embodiments, this concatenated vector may be fed into fully connected layers of the first RNN framework.

At step/operation 708, the predictive analysis engine 110 generates a fall prediction data object using the fall prediction machine learning model. The fall prediction machine learning model may be configured to generate the fall prediction data object utilizing an ensemble machine learning framework. The ensemble machine learning framework may be configured to generate a fall prediction data object based at least in part on the numerical timeseries embedding, the categorical timeseries embedding, and the static embedding. In some embodiments, the ensemble machine learning framework may be configured to generate a fall prediction data object comprising a fall likelihood prediction, one or more fall cause predictions, and/or a fall timing prediction.

At step/operation 408, the predictive analysis engine 110 of the predictive data analysis computing entity 106 may perform one or more prediction-based actions based at least in part on the fall prediction data object. The one or more prediction-based actions may be based at least in part on the fall prediction data object as generated in step/operation 406. For example, the one or more prediction based actions may comprise transmitting a fall prediction notification describing the fall prediction data object to one or more external computing entities 102, such as an edge client device. In some embodiments, the edge client computing entity may be configured to present one or more sensory notifications to an end user of the edge client device computing entity based at least in part on the fall prediction notification. The one or more sensory notifications may comprise one or more audiovisual notifications, one or more haptic notifications, and one or more electrical impulses. For example, if the fall prediction notification describes that the user is likely to experience a fall due to not taking his/her medication, the edge client computing entity may present a sensory notification comprising an audiovisual notification reminding the user described by the fall prediction notification to take his/her medication. In some embodiments, edge client computing entity may cause one or more haptic notifications and/or electrical impulses to occur to attempt to prevent a user fall or lessen the severity of a user fall moments from when the fall is predicted to occur.

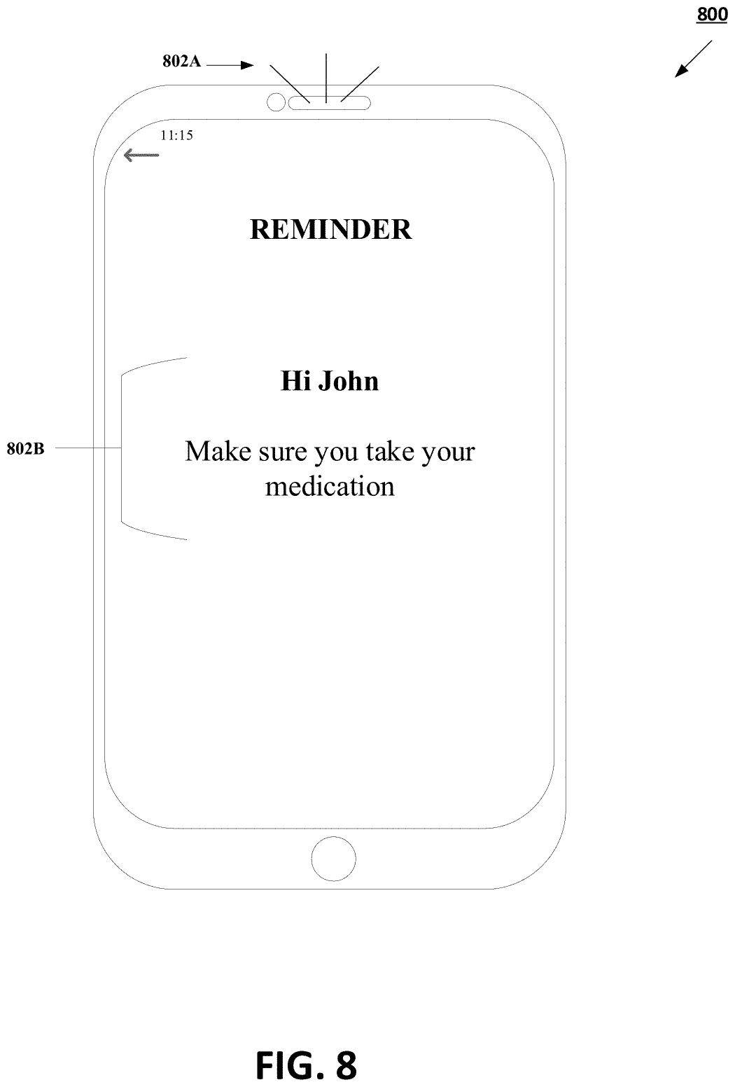

An operational example of an audiovisual sensory notification 800 presented to an end user on an edge client computing entity is depicted in FIG. 8. As depicted in FIG. 8, one or more audiovisual notifications may be presented to an end user. In some embodiments, the end user may be the user described by the fall prediction notification or may be a user associated with the user described by the fall prediction notification, such as a family member, friend, caretaker, or the like. In FIG. 8, the edge client computing entity may be a mobile phone. The edge client computing device may receive the transmitted fall prediction notification describing the fall prediction data object and present one or more sensory notifications to the end user based at least in part on the fall prediction notification. For example, the edge client computing entity may receive the fall prediction notification describing a fall prediction data object indicating that an associated user is likely to suffer a fall due to not taking his/her medication. The edge client computing device may generate one or more sensory notifications to the end user of the edge client computing entity notifying the user to take his/her medication or to remind the user associated with the fall prediction notification to take his/her medication.

The edge client computing entity may generate the one or more sensory notifications as one or more audiovisual notifications 802A-802B. For example, the edge client computing entity may display a visual reminder on a display associated with the edge client computing entity reminding the end user to take his/her medication or to alert an end user that a particular user needs to take his/her medication. The edge client computing device may also transmit an audio reminder alerting the end user to take his/her medication or to alert an end user that an associated user needs to take his/her medication. In some embodiments, an end user may configure his/her audiovisual notification preferences such that an end user may control the presentation of the one or more audiovisual notification on one or more edge user computing devices.

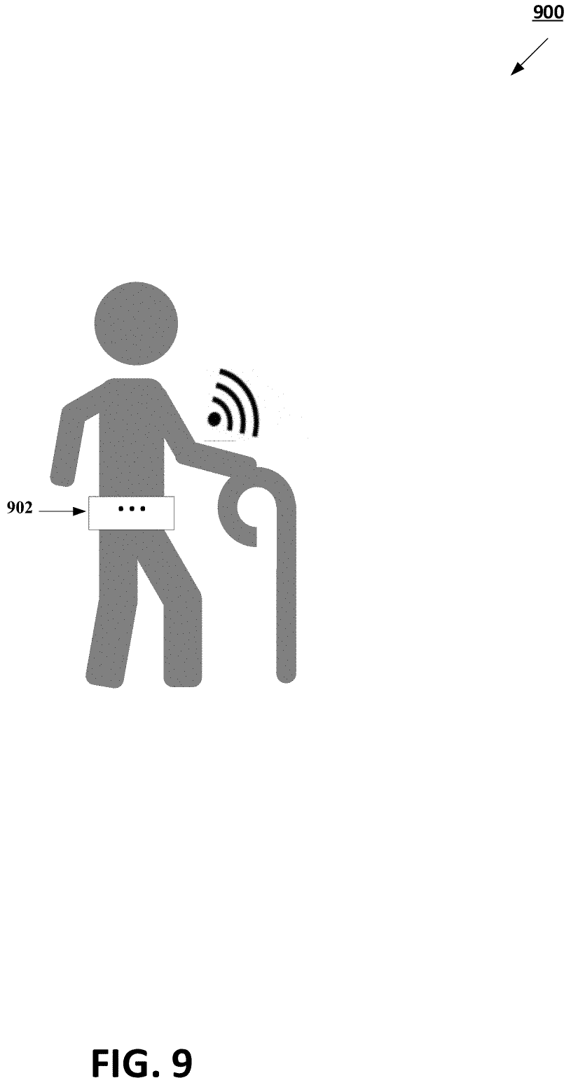

An operational example of a haptic sensory notification 900 presented to an end user on an edge client computing entity is depicted in FIG. 9. As depicted in FIG. 9, one or more haptic notifications may be presented to an end user and/or electrical impulses may be provided automatically. In some embodiments, the edge client computing device is a wearable device 902. In some embodiments, the wearable device 902 may be configured to provide haptic feedback and/or electrical impulse feedback, such as by using vibrations and/or one or more electrical impulses to cause stimulation of the end user's target muscle groups, thereby lessening the likelihood for a fall or reducing the severity of a fall in the event that such a fall occurs.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A computer-implemented method for dynamically generating a fall likelihood prediction for a user feature data object, the computer-implemented method comprising:
generating, using the one or more processors and by utilizing a fall prediction machine learning model that is configured to process a user feature data object, a fall prediction data object, wherein:
the fall prediction machine learning model is generated based at least in part on optimizing a custom loss model,
the custom loss model comprises a fall likelihood component and a fall cause component, and
the custom loss model is generated in accordance with a custom loss generation routine that comprises:
identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications;
generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications;
for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object;
generating the fall likelihood component based at least in part the fall likelihood loss values for the one or more training user feature data objects; and
generating the fall cause component based at least in part on the fall cause loss values for the one or more training user feature data objects; and
performing, using the one or more processors, one or more prediction-based actions based at least in part on the fall likelihood prediction.

2. The computer-implemented method of claim 1, wherein the fall prediction data object describes: (i) a fall likelihood prediction, and (ii) one or more fall cause predictions.

3. The computer-implemented method of claim 2, wherein the fall prediction data object further describes, in the instance where the fall likelihood prediction is the affirmative true likelihood prediction, a fall timing prediction.

4. The computer-implemented method of claim 1, wherein:
the user feature data object comprises one or more numerical timeseries feature data fields, one or more categorical timeseries feature data fields, and one or more static feature data fields; and
the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall likelihood prediction based at least in part on the numerical timeseries embedding, the categorical timeseries embedding, and the static embedding.

5. The computer-implement method of claim 1, wherein performing the one or more prediction-based actions comprises transmitting a fall prediction notification describing the fall prediction data object to an edge client computing entity, and
the edge client computing entity is configured to present one or more sensory notifications to an end user of the edge client computing based at least in part on the fall prediction notification.

6. The computer-implemented method of claim 6, wherein the sensory notifications comprise at least one of: (i) one or more audiovisual notifications, and (iii) one or more electrical pulse notifications.

7. The computer-implemented method of claim 1, wherein:
the fall prediction machine learning model has fewer parameters as compared to a trained teacher fall prediction machine learning model; and
the fall prediction machine learning model is trained based at least in part on a distillation loss, wherein the distillation loss comprises a custom loss generated by a custom loss model and a distillation loss score, and wherein the distillation loss score is based at least in part on one or more teacher outputs from the teacher fall prediction machine learning model, one or more inferred outputs of the fall prediction machine learning model, a ground truth fall likelihood and a ground truth fall cause.

8. An apparatus for dynamically generating a fall likelihood prediction for a user feature data object, the apparatus comprising at least one processor and at least one memory including program code, the at least one memory and the program code configured to, with the processor, cause the apparatus to at least:
generate, using a fall prediction machine learning model that is configured to process a user feature data object, a fall prediction data object, wherein:
the fall prediction machine learning model is generated based at least in part on optimizing a custom loss model,
the custom loss model comprises a fall likelihood component and a fall cause component, and
the custom loss model is generated in accordance with a custom loss generation routine that comprises:
identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications;
generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications;
for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object;
generating the fall likelihood component based at least in part the fall likelihood loss values for the one or more training user feature data objects; and
generating the fall cause component based at least in part on the fall cause loss values for the one or more training user feature data objects; and
perform one or more prediction-based actions based at least in part on the fall likelihood prediction.

9. The apparatus of claim 8, wherein the fall prediction data object describes: (i) a fall likelihood prediction, and (ii) one or more fall cause predictions.

10. The apparatus of claim 9, wherein the fall prediction data object further describes, in the instance where the fall likelihood prediction is the affirmative true likelihood prediction, a fall timing prediction.

11. The apparatus of claim 8, wherein:
the user feature data object comprises one or more numerical timeseries feature data fields, one or more categorical timeseries feature data fields, and one or more static feature data fields; and
the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall likelihood prediction based at least in part on the numerical timeseries embedding, the categorical timeseries embedding, and the static embedding.

12. The apparatus of claim 8, wherein performing the one or more prediction-based actions comprises transmitting a fall prediction notification describing the fall prediction data object to an edge client computing entity, and
the edge client computing entity is configured to present one or more sensory notifications to an end user of the edge client computing based at least in part on the fall prediction notification.

13. The apparatus of claim 12, wherein the sensory notifications comprise at least one of: (i) one or more audiovisual notifications, and (iii) one or more electrical pulse notifications.

14. The apparatus of claim 8, wherein:
the fall prediction machine learning model has fewer parameters as compared to a trained teacher fall prediction machine learning model; and
the fall prediction machine learning model is trained based at least in part on a distillation loss, wherein the distillation loss comprises a custom loss generated by a custom loss model and a distillation loss score, wherein the distillation loss score is based at least in part on one or more teacher outputs from the teacher fall prediction machine learning model, one or more inferred outputs of the fall prediction machine learning model, a ground truth fall likelihood and a ground truth fall cause.

15. A computer program product for dynamically generating a fall likelihood prediction for a user feature data object, the computer program product comprising at least one non-transitory computer-readable storage medium having computer-readable program code portions stored therein, the computer-readable program code portions configured to:
generate, using a fall prediction machine learning model that is configured to process a user feature data object, a fall prediction data object, wherein:
the fall prediction machine learning model is generated based at least in part on optimizing a custom loss model,
the custom loss model comprises a fall likelihood component and a fall cause component, and
the custom loss model is generated in accordance with a custom loss generation routine that comprises:
identifying one or more training user feature data objects, wherein: (i) the one or more training user feature data objects are associated with one or more ground-truth fall predictions, and (ii) each ground-truth fall prediction for a training user feature data object describes: (a) a ground-truth fall likelihood prediction, and (b) one or more ground-truth fall cause indications;
generating, by utilizing the fall prediction machine learning model, one or more inferred fall predictions for the one or more training user feature data objects, wherein each inferred fall prediction for a training user feature data object describes: (i) an inferred fall likelihood prediction, and (ii) one or more inferred fall cause indications;
for each training user feature data object, generating: (i) a fall likelihood loss value based at least in part on the ground-truth fall likelihood prediction for the training user feature data object and the inferred fall likelihood prediction for the training user feature data object, and (ii) one or more fall cause loss values based at least in part on the one or more ground-truth fall cause indications for the training user feature data object and the one or more inferred fall cause indications for the user feature data object;
generating the fall likelihood component based at least in part the fall likelihood loss values for the one or more training user feature data objects; and
generating the fall cause component based at least in part on the fall cause loss values for the one or more training user feature data objects; and
perform one or more prediction-based actions based at least in part on the fall likelihood prediction.

16. The computer program product of claim 15, wherein the fall prediction data object describes: (i) a fall likelihood prediction, and (ii) one or more fall cause predictions.

17. The computer program product of claim 15, wherein the fall prediction data object further describes, in the instance where the fall likelihood prediction is the affirmative true likelihood prediction, a fall timing prediction.

18. The computer program product of claim 15, wherein:
the user feature data object comprises one or more numerical timeseries feature data fields, one or more categorical timeseries feature data fields, and one or more static feature data fields; and
the fall prediction machine learning model comprises: (i) a first recurrent neural network (RNN) framework that is configured to process the one or more numerical timeseries feature data fields to generate a numerical timeseries embedding for the user feature data object, (ii) a second RNN framework that is configured to process the one or more categorical timeseries feature data fields to generate a categorical timeseries embedding for the user feature data object, (iii) a fully connected neural network framework that is configured to process the one or more static feature data fields to generate a static embedding for the user feature data object, (iv) an ensemble machine learning framework that is configured to generate the fall likelihood prediction based at least in part on the numerical timeseries embedding, the categorical timeseries embedding, and the static embedding.

19. The computer program product of claim 15, wherein performing the one or more prediction-based actions comprises transmitting a fall prediction notification describing the fall prediction data object to an edge client computing entity, and
the edge client computing entity is configured to present one or more sensory notifications to an end user of the edge client computing based at least in part on the fall prediction notification.

20. The computer program product of claim 15, wherein:
the fall prediction machine learning model has fewer parameters as compared to a trained teacher fall prediction machine learning model; and
the fall prediction machine learning model is trained based at least in part on a distillation loss, wherein the distillation loss comprises a custom loss generated by a custom loss model and a distillation loss score, and wherein the distillation loss score is based at least in part on one or more teacher outputs from the teacher fall prediction machine learning model, one or more inferred outputs of the fall prediction machine learning model, a ground truth fall likelihood and a ground truth fall cause.
